(19) United States
(12) Patent Application Publication
(10) Pub. No.: US 2013/0165793 A1
(43) Pub. Date: Jun. 27, 2013

(54) PROVIDING DOPPLER INFORMATION OF TARGET OBJECT BASED ON VECTOR DOPPLER IN ULTRASOUND SYSTEM

(71) Applicant: SAMSUNG MEDISON CO., LTD, Gangwon-do (KR)

(72) Inventor: Deok Gon KIM, Seoul (KR)

(73) Assignee: SAMSUNG MEDISON CO., LTD, Gangwon-do (KR)

(21) Appl. No.: 13/728,526

(22) Filed: Dec. 27, 2012

(30) Foreign Application Priority Data
Dec. 27, 2011 (KR) 10-2011-0143848

(51) Int. Cl. A61B 8/08

(52) U.S. Cl. CPC A61B 8/488
USPC 600/453

(57) ABSTRACT

There are provided embodiments for providing Doppler information of a target object by using vector Doppler. In one embodiment, by way of non-limiting example, an ultrasound system comprises: a processing unit configured to form vector information of a target object based on ultrasound data corresponding to the target object, the processing unit being further configured to form motion information corresponding to motion of the target object based on the vector information, wherein the motion information includes at least one of a Doppler spectrum image and a Doppler sound.

### CROSS-REFERENCE TO RELATED APPLICATIONS

The present application claims priority from Korean Patent Application No. 10-2011-0143848 filed on Dec. 27, 2011, the entire subject matter of which is incorporated herein by reference.

### TECHNICAL FIELD

The present disclosure generally relates to ultrasound systems, and more particularly to providing the Doppler information of a target object by using vector Doppler in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of target objects (e.g., human organs).

The ultrasound system may provide ultrasound images of various modes including a brightness mode image representing reflection coefficients of ultrasound signals (i.e., ultrasound echo signals) reflected from a target object of a living body with a two-dimensional image, a Doppler mode image representing velocity of a moving target object with spectral Doppler by using a Doppler effect, a color Doppler mode image representing velocity of the moving target object with colors by using the Doppler effect, an elastic image representing mechanical characteristics of tissues before and after applying compression thereto and the like.

The ultrasound system may transmit the ultrasound signals to the living body and receive the ultrasound echo signals from the living body to form Doppler signals corresponding to a region of interest, which is set on the brightness mode image. The ultrasound system may further form the color Doppler mode image representing the velocity of the moving target object with colors based on the Doppler signals. In particular, the color Doppler image may represent the motion of the target object (e.g., blood flow) with the colors. The color Doppler image may be used to diagnose disease of a blood vessel, a heart and the like. However, it is difficult to represent an accurate motion of the target object (e.g., blood flow) since the respective colors indicated by a motion value is a function of the velocity of the target object, which moves forward in a transmission direction of the ultrasound signals and moves backward in the transmission direction of the ultrasound signals.

To resolve this problem, a vector Doppler method capable of obtaining the velocity and direction of the blood flow is used. A cross beam-based method of the vector Doppler method may acquire velocity magnitude components from at least two different directions, and combine the velocity magnitude components to detect vector information having a two-dimensional or three-dimensional direction information and a magnitude information.

### SUMMARY

There are provided embodiments for providing the Doppler information of a target object by using vector Doppler.

In one embodiment, by way of non-limiting example, an ultrasound system comprises: a processing unit configured to form vector information of a target object based on ultrasound data corresponding to the target object, and form Doppler information corresponding to motion of the target object based on the vector information, wherein the Doppler information includes at least one of a Doppler spectrum image and a Doppler sound.

In another embodiment, there is provided a method of providing Doppler information, comprising: a) forming vector information of a target object based on ultrasound data corresponding to the target object; and b) forming Doppler information corresponding to motion of the target object based on the vector information, wherein the Doppler information includes at least one of a Doppler spectrum image and a Doppler sound.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a schematic diagram showing an example of a brightness mode image and a region of interest.

FIG. 3 is a block diagram showing an illustrative embodiment of an ultrasound data acquiring unit.

FIGS. 4 to 7 are schematic diagrams showing examples of transmission directions and reception directions.

FIG. 8 is a schematic diagram showing an example of sampling data and pixels of an ultrasound image.

FIGS. 9 to 12 are schematic diagrams showing examples of performing a reception beam-forming.

FIG. 13 is a schematic diagram showing an example of setting weights.

FIG. 14 is a schematic diagram showing an example of setting a sampling data set.

FIG. 15 is a flow chart showing a process of forming the Doppler information of a target object.

FIG. 16 is a schematic diagram showing an example of the transmission directions, the reception directions, the vector information and an over-determined problem.

FIG. 17 is a schematic diagram showing an example of a Doppler spectrum image.

FIG. 18 is a schematic diagram showing an example of a Doppler spectrum image and a Doppler sound.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

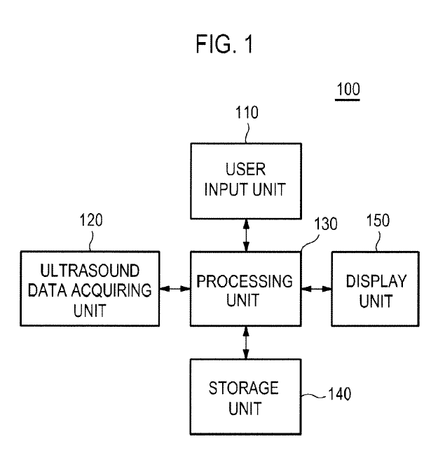

Referring to FIG. 1, an ultrasound system 100 in accordance with an illustrative embodiment is shown. As depicted herein, the ultrasound system 100 may include a user input unit 110.

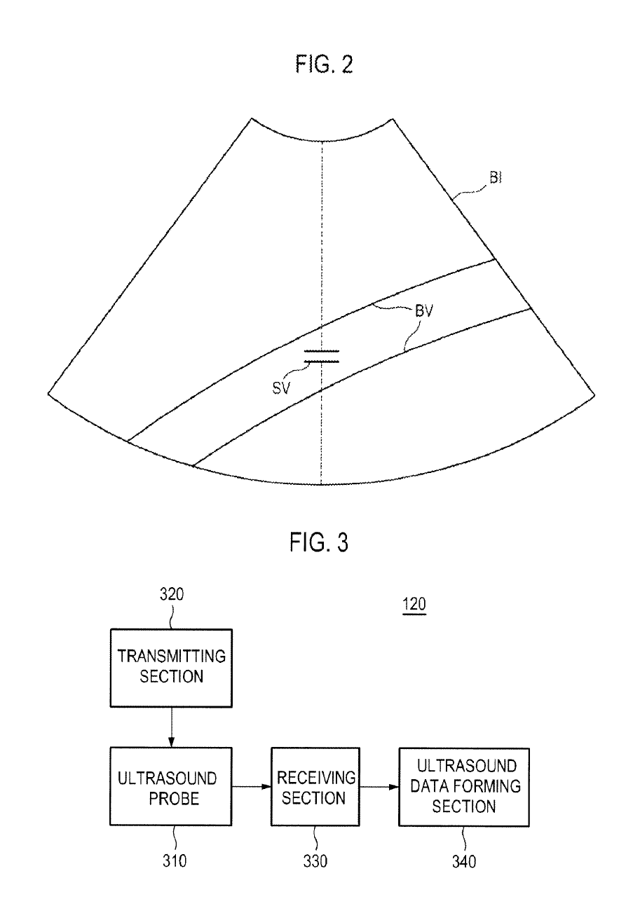

The user input unit 110 may be configured to receive input information from a user. In one embodiment, the input information may include information for setting a region of interest SV on a brightness mode image BI, as shown in FIG. 2. However, it should be noted herein that the input information may not be limited thereto. The region of interest SV may include a sample volume. In FIG. 2, the reference numeral BV represents a blood vessel. The user input unit 110 may include a control panel, a track ball, a touch screen, a mouse, a keyboard and the like.

The ultrasound system 100 may further include an ultrasound data acquiring unit 120. The ultrasound data acquiring unit 120 may be configured to transmit ultrasound signals to a living body. The living body may include target objects (e.g., blood vessel, heart, blood flow, etc). The ultrasound data acquiring unit 120 may be further configured to receive ultrasound signals (i.e., ultrasound echo signals) from the living body to acquire ultrasound data corresponding to an ultrasound image.

FIG. 3 is a block diagram showing an illustrative embodiment of the ultrasound data acquiring unit. Referring to FIG. 3, the ultrasound data acquiring unit 120 may include an ultrasound probe 310.

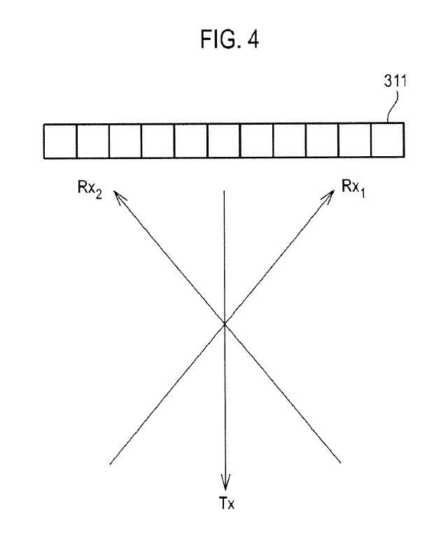

The ultrasound probe 310 may include a plurality of elements 311 (see FIG. 4) for reciprocally converting between ultrasound signals and electrical signals. The ultrasound probe 310 may be configured to transmit the ultrasound signals to the living body. The ultrasound signals transmitted from the ultrasound probe 310 may be plane wave signals that the ultrasound signals are not focused at a focusing point or focused signals that the ultrasound signals are focused at the focusing point. However, it should be noted herein that the ultrasound signals may not be limited thereto. The ultrasound probe 310 may be further configured to receive the ultrasound echo signals from the living body to output electrical signals (hereinafter referred to as “reception signals”). The reception signals may be analog signals. The ultrasound probe 310 may include a convex probe, a linear probe and the like,

The ultrasound data acquiring unit 120 may further include a transmitting section 320. The transmitting section 320 may be configured to control the transmission of the ultrasound signals. The transmitting section 320 may be further configured to generate electrical signals (hereinafter referred to as “transmission signals”) in consideration of the elements 311.

In one embodiment, the transmitting section 320 may be configured to generate transmission signals (hereinafter referred to as “brightness mode transmission signals”) for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output reception signals (hereinafter referred to as “brightness mode reception signals”).

The transmitting section 320 may be further configured to generate transmission signals (hereinafter referred to as “Doppler mode transmission signals”) corresponding to an ensemble number in consideration of the elements 311 and at least one transmission direction of the ultrasound signals (i.e., transmission beam). Thus, the ultrasound probe 310 may be configured to convert the Doppler mode transmission signals provided from the transmitting section 320 into the ultrasound signals, transmit the ultrasound signals to the living body in the at least one transmission direction, and receive the ultrasound echo signals from the living body to output reception signals (hereinafter referred to as “Doppler mode reception signals”). The ensemble number may represent the number of transmitting and receiving the ultrasound signals.

As one example, the transmitting section 320 may be configured to generate the Doppler mode transmission signals corresponding to the ensemble number in consideration of a transmission direction Tx and the elements 311, as shown in FIG. 4. The transmission direction may be one of a direction (0 degree) perpendicular to a longitudinal direction of the elements 311 to a maximum steering direction of the transmission beam.

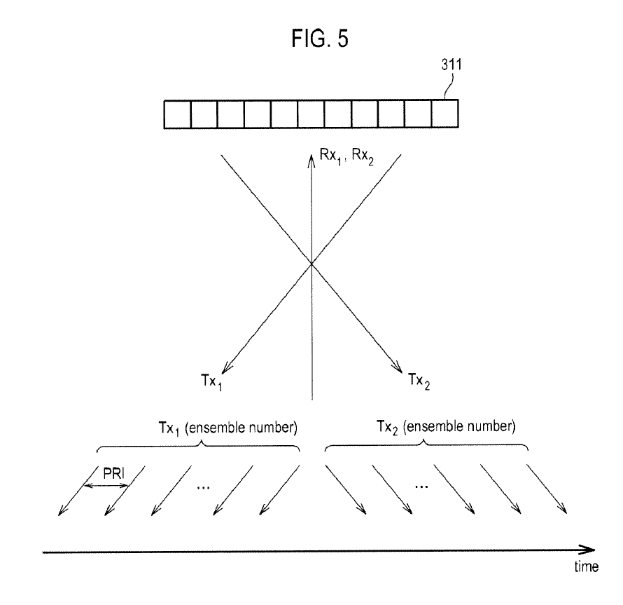

As another example, the transmitting section 320 may be configured to generate first Doppler mode transmission signals corresponding to the ensemble number in consideration of a first transmission direction Tx₁ and the elements 311, as shown in FIG. 5. Thus, the ultrasound probe 310 may be configured to convert the first Doppler mode transmission signals provided from the transmitting section 320 into the ultrasound signals, transmit the ultrasound signals to the living body in the first transmission direction Tx₁, and receive the ultrasound echo signals from the living body to output first Doppler mode reception signals. The transmitting section 320 may be further configured to generate second Doppler mode transmission signals corresponding to the ensemble number in consideration of a second transmission direction Tx₂ and the elements 311, as shown in FIG. 5. Thus, the ultrasound probe 310 may be configured to convert the second Doppler mode transmission signals provided from the transmitting section 320 into the ultrasound signals, transmit the ultrasound signals to the living body in the second transmission direction Tx₂, and receive the ultrasound echo signals from the living body to output second Doppler mode reception signals. In FIG. 5, a reference numeral PRI represents a pulse repeat interval.

In another embodiment, the transmitting section 320 may be configured to generate the brightness mode transmission signals for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output the brightness mode reception signals.

The transmitting section 320 may be further configured to generate the Doppler mode transmission signals corresponding to the ensemble number in consideration of the at least one transmission direction and the elements 311. Thus, the ultrasound probe 310 may be configured to convert the Doppler mode transmission signals provided from the transmitting section 320 into the ultrasound signals, transmit the ultrasound signals to the living body, and receive the ultrasound echo signals from the living body to output the Doppler mode reception signals. The ultrasound signals may be transmitted in an interleaved transmission scheme. The interleaved transmission scheme will be described below in detail.

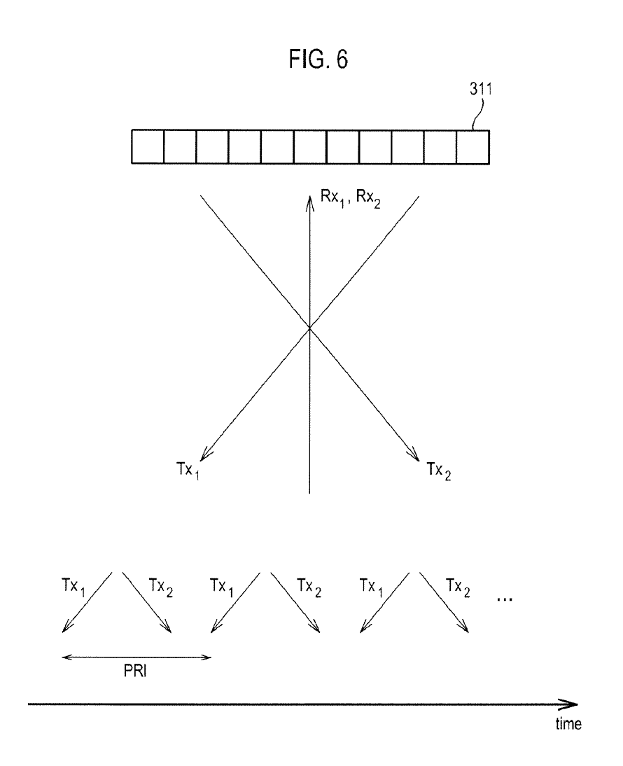

For example, the transmitting section 320 may be configured to generate the first Doppler mode transmission signals in consideration of the first transmission direction Tx₁ and the elements 311, as shown in FIG. 6. Thus, the ultrasound probe 310 may be configured to convert the first Doppler mode transmission signals provided from the transmitting section 320 into the ultrasound signals, and transmit the ultrasound signals to the living body in the first transmission direction Tx₁. Then, the transmitting section 320 may be further configured to generate the second Doppler mode transmission signals in consideration of the second transmission direction Tx₂ and the elements 311, as shown in FIG. 6. Thus, the ultrasound probe 310 may be configured to convert the second Doppler mode transmission signals provided from the transmitting section 320 into the ultrasound signals, and transmit the ultrasound signals to the living body in the second transmission direction Tx₂. The ultrasound probe 310 may be further configured to receive the ultrasound echo signals (i.e., ultrasound echo signals corresponding to first Doppler mode transmission signals) from the living body to output the first Doppler mode reception signals. The ultrasound probe may be further configured to receive the ultrasound echo signals (i.e., ultrasound echo signals corresponding to second Doppler mode transmission signals) from the living body to output the second Doppler mode reception signals.

Thereafter, the transmitting section 320 may be configured to generate the first Doppler mode transmission signals based on the pulse repeat interval, as shown in FIG. 6. Thus, the ultrasound probe 310 may be configured to convert the first Doppler mode transmission signals provided from the transmitting section 320 into the ultrasound signals, and transmit the ultrasound signals to the living body in the first transmission direction Tx₁. Then, the transmitting section 320 may be further configured to generate the second Doppler mode transmission signals based on the pulse repeat interval, as shown in FIG. 6. Accordingly, the ultrasound probe 310 may be configured to convert the second Doppler mode transmission signals provided from the transmitting section 320 into the ultrasound signals, and transmit the ultrasound signals to the living body in the second transmission direction Tx₂. The ultrasound probe 310 may be further configured to receive the ultrasound echo signals (i.e., ultrasound echo signals corresponding to first Doppler mode transmission signals) from the living body to output the first Doppler mode reception signals. The ultrasound probe 310 may be further configured to receive the ultrasound echo signals (i.e., ultrasound echo signals corresponding to second Doppler mode transmission signals) from the living body to output the second Doppler mode reception signals.

As described above, the transmitting section 320 may be configured to generate the first Doppler mode transmission signals and the second Doppler mode transmission signals corresponding to the ensemble number.

In yet another embodiment, the transmitting section 320 may be configured to generate the brightness mode transmission signals for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output the brightness mode reception signals.

The transmitting section 320 may be further configured to generate the Doppler mode transmission signals corresponding to the ensemble number in consideration of the at least one transmission direction and the elements 311. Thus, the ultrasound probe 310 may be configured to convert the Doppler mode transmission signals provided from the transmitting section 320 into the ultrasound signals, transmit the ultrasound signals to the living body in the at least one transmission direction, and receive the ultrasound echo signals from the living body to output the Doppler mode reception signals. The ultrasound signals may be transmitted according to the pulse repeat interval.

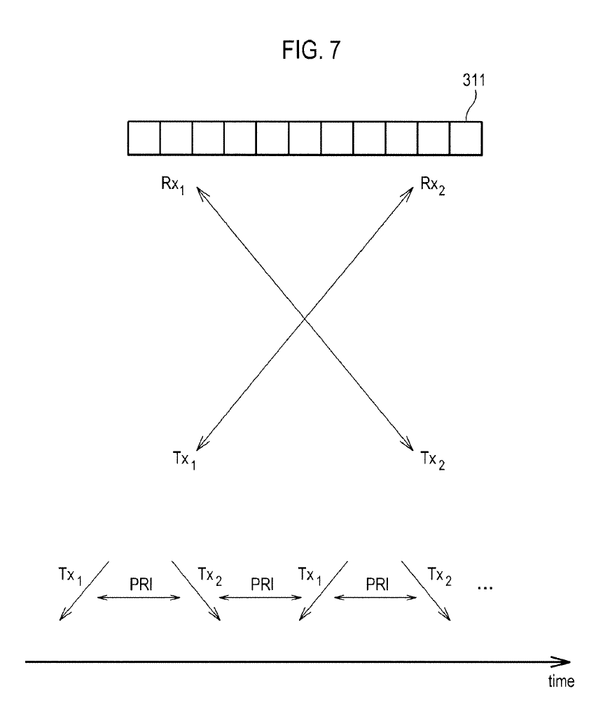

For example, the transmitting section 320 may be configured to generate the first Doppler mode transmission signals in consideration of the first transmission direction Tx₁ and the elements 311 based on the pulse repeat interval, as shown in FIG. 7. Thus, the ultrasound probe 310 may be configured to convert the first Doppler mode transmission signals provided from the transmitting section 320 into the ultrasound signals, transmit the ultrasound signals to the living body in the first transmission direction Tx₁, and receive the ultrasound echo signals from the living body to output the first Doppler mode reception signals. The transmitting section 320 may be further configured to generate the second Doppler mode transmission signals in consideration of the second transmission direction Tx₂ and the element 311 based on the pulse repeat interval, as shown in FIG. 7. Thus, the ultrasound probe 310 may be configured to convert the second Doppler mode transmission signals provided from the transmitting section 320 into the ultrasound signals, transmit the ultrasound signals to the living body in the second transmission direction Tx₂, and receive the ultrasound echo signals from the living body to output the second Doppler mode reception signals.

As described above, the transmitting section 320 may be configured to generate the first Doppler mode transmission signals and the second Doppler mode transmission signals corresponding to the ensemble number based on the pulse repeat interval.

Referring back to FIG. 3, the ultrasound data acquiring unit 120 may further include a receiving section 330. The receiving section 330 may be configured to perform an analog-digital conversion upon the reception signals provided from the ultrasound probe 310 to form sampling data. The receiving section 330 may be further configured to perform a reception beam-forming upon the sampling data in consideration of the elements 311 to form reception-focused data. The reception beam-forming will be described below in detail.

In one embodiment, the receiving section 330 may be configured to perform the analog-digital conversion upon the brightness mode reception signals provided from the ultrasound probe 310 to form sampling data (hereinafter referred to as “brightness mode sampling data”). The receiving section 330 may be further configured to perform the reception beam-forming upon the brightness mode sampling data to form reception-focused data (hereinafter referred to as “brightness mode reception-focused data”).

The receiving section 330 may be further configured to perform the analog-digital conversion upon the Doppler mode reception signals provided from the ultrasound probe 310 to form sampling data (hereinafter referred to as “Doppler mode sampling data”). The receiving section 330 may be further configured to perform the reception beam-forming upon the Doppler mode sampling data to form reception-focused data (hereinafter referred to as “Doppler mode reception-focused data”) corresponding to the at least one reception direction of the ultrasound echo signals (i.e., reception beam).

As one example, the receiving section 330 may be configured to perform the analog-digital conversion upon the Doppler mode reception signals provided from the ultrasound probe 310 to form the Doppler mode sampling data. The receiving section 330 may be further configured to perform the reception beam-forming upon the Doppler mode sampling data to form first Doppler mode reception-focused data corresponding to the first reception direction Rx₁ and second Doppler mode reception-focused data corresponding to the second reception direction Rx₂, as shown in FIG. 4.

As another example, the receiving section 330 may be configured to perform the analog-digital conversion upon the first Doppler mode reception signals provided from the ultrasound probe 310 to form first Doppler mode sampling data corresponding to the first transmission direction Tx₁, as shown in FIG. 5. The receiving section 330 may be further configured to perform the reception beam-forming upon the first Doppler mode sampling data to form the first Doppler mode reception-focused data corresponding to the first reception direction Rx₁. The receiving section 330 may be also configured to perform the analog-digital conversion upon the second Doppler mode reception signals provided from the ultrasound probe 310 to form second Doppler mode sampling data corresponding to the second transmission direction Tx₂, as shown in FIG. 5. The receiving section 330 may be further configured to perform the reception beam-forming upon the second Doppler mode sampling data to form the second Doppler mode reception-focused data corresponding to the second reception direction Rx₂. If the reception direction is perpendicular to the elements 311 of the ultrasound probe 310, then a maximum aperture size may be used.

The reception beam-forming may be described with reference to the accompanying drawings.

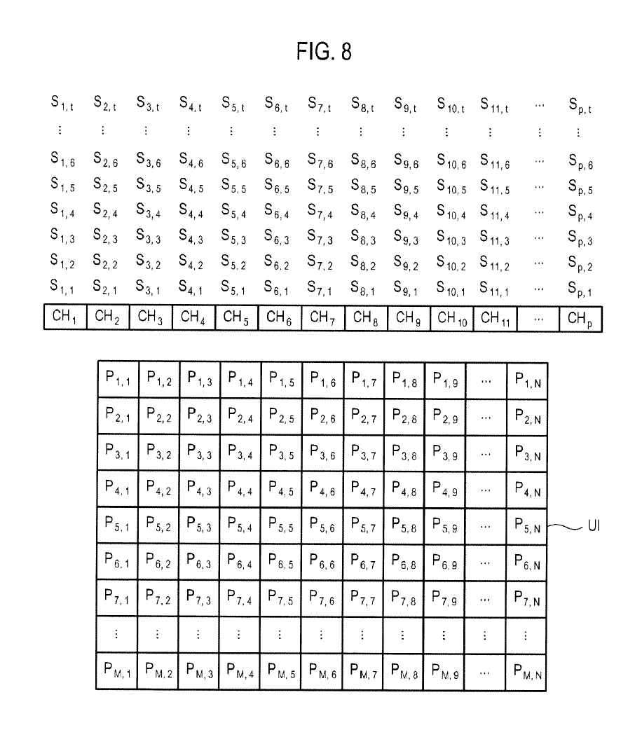

In one embodiment, the receiving section 330 may be configured to perform the analog-digital conversion upon the reception signals provided through a plurality of channels CHₖ, wherein 1≦k≦N, from the ultrasound probe 310 to form sampling data S_{i,j} wherein the i and j are a positive integer, as shown in FIG. 8. The sampling data S_{i,j} may be stored in a storage unit 140. The receiving section 330 may be further configured to detect pixels corresponding to the sampling data based on positions of the elements 311 and positions (orientation) of pixels of the ultrasound image UI with respect to the elements 311. That is, the receiving section 330 may select the pixels, which the respective sampling data are used as pixel data thereof, during the reception beam-forming based on the positions of the elements 311 and the orientation of the respective pixels of the ultrasound image UI with respect to the elements 311. The receiving section 330 may be configured to cumulatively assign the sampling data corresponding to the selected pixels as the pixel data.

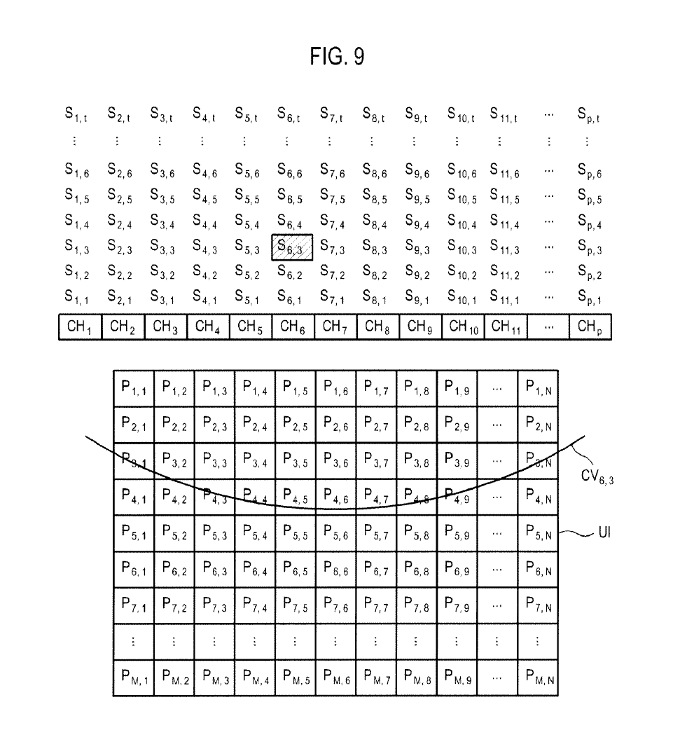

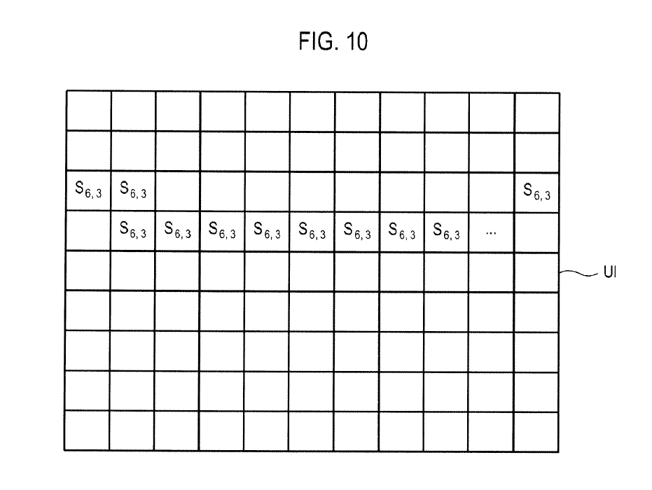

For example, the receiving section 330 may be configured to set a curve (hereinafter referred to as “reception beam-forming curve”) CV_{6,3} for selecting pixels, which the sampling data S_{6,3} are used as the pixel data thereof, during the reception beam-forming based on the positions of the elements 311 and the orientation of the respective pixels of the ultrasound image UI with respect to the elements 311, as shown in FIG. 9. The receiving section 330 may be further configured to detect the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} corresponding to the reception beam-forming curve CV_{6,3} from the pixels P_{a,b} of the ultrasound image UI, wherein 1≦a≦M, 1≦b≦N. That is, the receiving section 330 may select the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} on which the reception beam-forming curve CV_{6,3} passes among the pixels P_{a,b} of the ultrasound image UI. The receiving section 330 may be also configured to assign the sampling data S_{6,3} to the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}, as shown in FIG. 10.

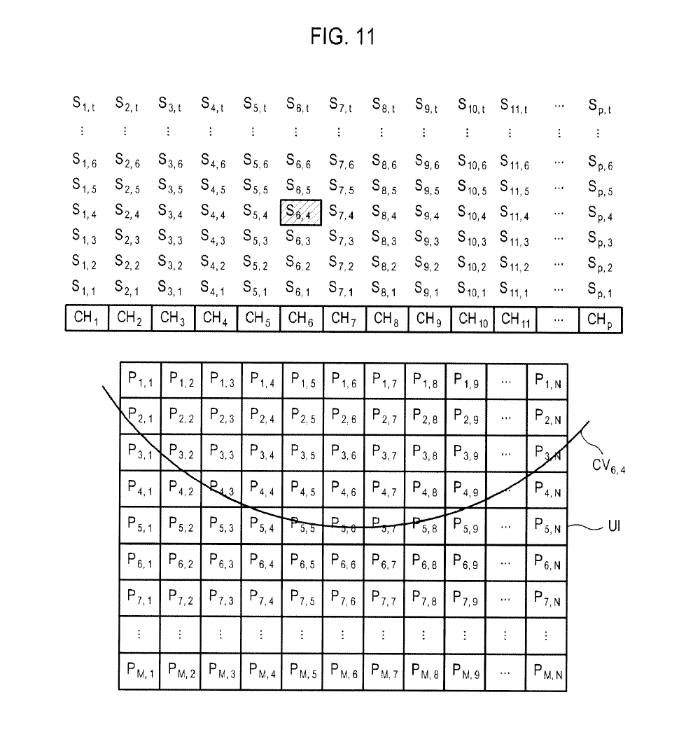

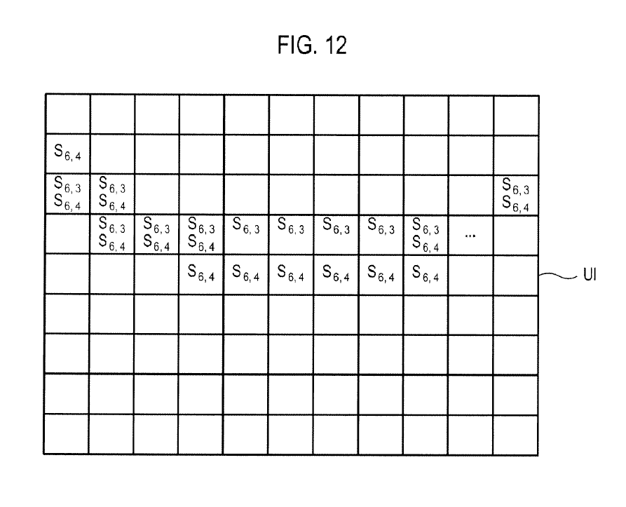

Thereafter, the receiving section 330 may be configured to set a reception beam-forming curve CV_{6,4} for selecting pixels, which the sampling data S_{6,4} are used as the pixel data thereof, during the reception beam-forming based on the positions of the elements 311 and the orientation of the respective pixels of the ultrasound image UI with respect to the elements 311, as shown in FIG. 11. The receiving section 330 may be further configured to detect the pixels P_{2,1}, P_{3,1}, P_{3,2}, P_{4,1}, P_{4,3}, P_{4,4}, P_{5,4}, P_{5,5}, P_{5,6}, P_{5,7}, P_{5,8}, P_{4,9}, P_{5,9}, . . . P_{4,N}, P_{3,N} corresponding to the reception beam-forming curve CV_{6,4} from the pixels P_{a,b} of the ultrasound image UI. That is, the receiving section 330 may select the pixels P_{2,1}, P_{3,1}, P_{3,2}, P_{4,2}, P_{4,3}, P_{4,4}, P_{5,4}, P_{5,5}, P_{5,6}, P_{5,7}, P_{5,8}, P_{4,9}, P_{5,9}, . . . P_{4,N}, P_{3,N} on which the reception beam-forming curve CV_{6,4} passes among the pixels P_{a,b} of the ultrasound image UI. The receiving section 330 may be further configured to assign the sampling data S_{6,4} to the selected pixels P_{2,1}, P_{3,1}, P_{3,2}, P_{4,2}, P_{4,3}, P_{4,4}, P_{5,4}, P_{5,5}, P_{5,6}, P_{5,7}, P_{5,8}, P_{5,9}, . . . P_{4,N}, P_{3,N}, as shown in FIG. 12. In this way, the respective sampling data, which are used as the pixel data, may be cumulatively assigned to the pixels as the pixel data.

The receiving section 330 may be configured to perform the reception beam-forming (i.e., summing) upon the sampling data, which are cumulatively assigned to the respective pixels P_{a,b} of the ultrasound image UI to form the reception-focused data.

In another embodiment, the receiving section 330 may be configured to perform the analog-digital conversion upon the reception signals provided through the plurality of channels CHₖ from the ultrasound probe 310 to form the sampling data S_{i,j}, as shown in FIG. 8. The sampling data S_{i,j} may be stored in the storage unit 140. The receiving section 330 may be further configured to detect pixels corresponding to the sampling data based on the positions of the elements 311 and the position (orientation) of the pixels of the ultrasound image UI with respect to the elements 311. That is, the receiving section 330 may select the pixels, which the respective sampling data are used as the pixel data thereof, during the reception beam-forming based on the positions of the elements 311 and the orientation of the respective pixels of the ultrasound image UI with respect to the elements 311. The receiving section 330 may be configured to cumulatively assign the sampling data corresponding to the selected pixels as the pixel data. The receiving section 330 may be further configured to determine pixels existing in the same column among the selected pixels. The receiving section 330 may be also configured to set weights corresponding to the respective determined pixels. The receiving section 330 may be additionally configured to apply the weights to the sampling data of the respective pixels.

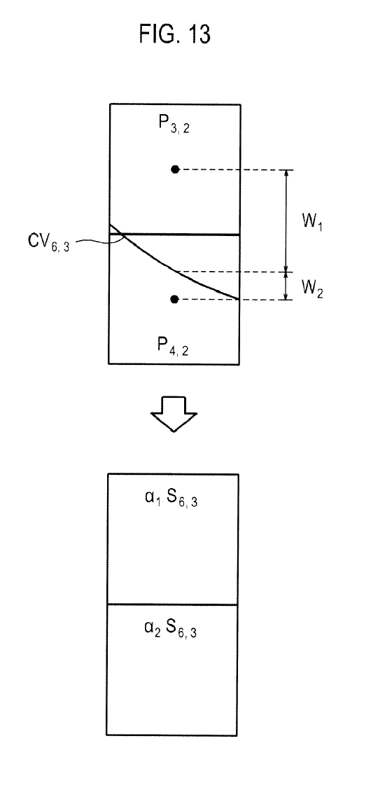

For example, the receiving section 330 may be configured to set the reception beam-forming curve CV_{6,3} for selecting pixels, which the sampling data S_{6,3} are used as the pixel data thereof, during the reception beam-forming based on the positions of the elements 311 and the orientation of the respective pixels of the ultrasound image UI with respect to the elements 311, as shown in FIG. 9. The receiving section 330 may be further configured to detect the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} corresponding to the reception beam-forming curve CV_{6,3} from the pixels P_{a,b} of the ultrasound image UI, wherein 1≦a≦M, 1≦b≦N. That is, the receiving section 330 may select the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} on which the reception beam-forming curve CV_{6,3} passes among the pixels P_{a,b} of the ultrasound image UI. The receiving section 330 may be also configured to assign the sampling data S_{6,3} to the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}, as shown in FIG. 10. The receiving section 330 may be further configured to determine pixels P_{3,2} and P_{4,2}, which exist in the same column among the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}. The receiving section 330 may be further configured to calculate a distance W₁ from a center of the determined pixel P_{3,2} to the reception beam-forming curve CV_{6,3} and a distance W₂ from a center of the determined pixel P_{4,2} to the reception beam-forming curve CV_{6,3}, as shown in FIG. 13. The receiving section 330 may be additionally configured to set a first weight α₁ corresponding to the pixel P_{3,2} based on the distance W₁ and a second weight co corresponding to the pixel P_{4,2} based on the distance W₂. The first weight α₁ and the second weight α₂ may be set to be in proportional to or in inverse proportional to the calculated distances. The receiving section 330 may be further configured to apply the first weight α₁ to the sampling data S_{6,3} assigned to the pixel P_{3,2} and to apply the second weight α₂ to the sampling data S_{6,3} assigned to the pixel P_{4,2}. The receiving section 330 may be configured to perform the above process upon the remaining sampling data.

The receiving section 330 may be configured to perform the reception beam-forming upon the sampling data, which are cumulatively assigned to the respective pixels P_{a,b} of the ultrasound image UI to form the reception-focused data.

In yet another embodiment, the receiving section 330 may be configured to perform the analog-digital conversion upon the reception signals provided through the plurality of channels CHₖ from the ultrasound probe 310 to form the sampling data S_{i,j}, as shown in FIG. 8. The sampling data S_{i,j} may be stored in the storage unit 140. The receiving section 330 may be further configured to set a sampling data set based on the sampling data S_{i,j}. That is, The receiving section 330 may set the sampling data set for selecting pixels, which the sampling data S_{i,j} are used as the pixel data thereof, during the reception beam-forming.

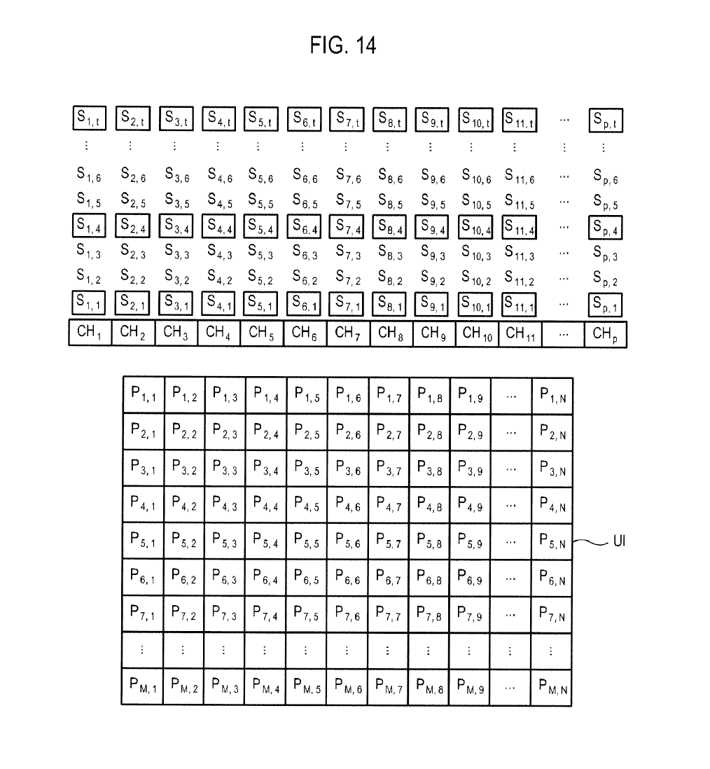

For example, the receiving section 330 may be configured to set the sampling data S_{1,1}, S_{1,4}, . . . S_{1,t}, S_{2,1}, S_{2,4}, . . . , S_{2,t}, . . . S_{p,t} as the sampling data set (denoted by a box) for selecting the pixels, which the sampling data S_{i,j} are used as the pixel data thereof, during the reception beam-forming, as shown in FIG. 14.

The receiving section 330 may be further configured to detect the pixels corresponding to the respective sampling data of the sampling data set based on the positions of the elements 311 and the positions (orientation) of the respective pixels of the ultrasound image UI with respect to the elements 311. That is, the receiving section 330 may select the pixels, which the respective sampling data of the sampling data set are used as the pixel data thereof, during the reception beam-forming based on the positions of the elements 311 and the orientation of the respective pixels of the ultrasound image UI with respect to the elements 311. The receiving section 330 may be further configured to cumulatively assign the sampling data to the selected pixels in the same manner with the above embodiments. The receiving section 330 may be also configured to perform the reception beam-forming upon the sampling data, which are cumulatively assigned to the respective pixels of the ultrasound image UI to form the reception-focused data.

In yet another embodiment, the receiving section 330 may be configured to perform a down-sampling upon the reception signals provided through the plurality of channels CHₖ from the ultrasound probe 310 to form down-sampling data. As described above, the receiving section 330 may be further configured to detect the pixels corresponding to the respective sampling data, based on the positions of the elements 311 and the positions (orientation) of the respective pixels of the ultrasound image UI with respect to the elements 311. That is, the receiving section 330 may select the pixels, which the respective sampling data are used as the pixel data thereof, during the reception beam-forming based on the positions of the elements 311 and the orientation of the pixels of the ultrasound image UI with respect to the elements 311. The receiving section 330 may be further configured to cumulatively assign the respective sampling data to the selected pixels in the same manner of the above embodiments. The receiving section 330 may be further configured to perform the reception beam-forming upon the sampling data, which are cumulatively assigned to the respective pixels of the ultrasound image UI to form the reception-focused data.

However, it should be noted herein that the reception beam-forming may not be limited thereto.

Referring back to FIG. 3, the ultrasound data acquiring unit 120 may further include an ultrasound data forming section 340. The ultrasound data forming section 340 may be configured to form the ultrasound data corresponding to the ultrasound image based on the reception-focused data provided from the receiving section 330. The ultrasound data forming section 340 may be further configured to perform a signal process (e.g., gain control, etc) upon the reception-focused data.

In one embodiment, the ultrasound data forming section 340 may be configured to form ultrasound data (hereinafter referred to as “brightness mode ultrasound data”) corresponding to the brightness mode image based on the brightness mode reception-focused data provided from the receiving section 330. The brightness mode ultrasound data may include radio frequency data.

The ultrasound data forming section 340 may be further configured to form ultrasound data (hereinafter referred to as “Doppler mode ultrasound data”) corresponding to the region of interest SV (i.e., Doppler spectrum image) based on the Doppler mode reception-focused data provided from the receiving section 330. The Doppler mode ultrasound data may include in-phase/quadrature data. However, it should be noted herein that the Doppler mode ultrasound data may not be limited thereto.

For example, the ultrasound data forming section 340 may form first Doppler mode ultrasound data based on the first Doppler mode reception-focused data provided from the receiving section 330. The ultrasound data forming section 340 may further form second Doppler mode ultrasound data based on the second Doppler mode reception-focused data provided from the receiving section 330.

Referring back to FIG. 1, the ultrasound system 100 may further include a processing unit 130 in communication with the user input unit 110 and the ultrasound data acquiring unit 120. The processing unit 130 may include a central processing unit, a microprocessor, a graphic processing unit and the like.

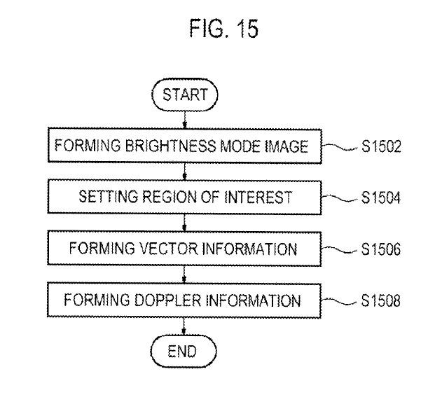

FIG. 15 is a flow chart showing a process of forming Doppler information of the target object. The processing unit 130 may be configured to form the brightness mode image BI based on the brightness mode ultrasound data provided from the ultrasound data acquiring unit 120, at step S1502 in FIG. 15. The brightness mode image BI may be displayed on a display unit (not shown) of an output unit 150. Thus, the user may set the region of interest SV on the brightness mode image BI by using the user input unit 110.

The processing unit 130 may be configured to set the region of interest SV on the brightness mode image BI based on the input information provided from the user input unit 110, at step S1504 in FIG. 15. Thus, the ultrasound data acquiring unit 120 may be configured to transmit the ultrasound signals to the living body and receive the ultrasound echo signals from the living body to acquire the Doppler mode ultrasound data, in consideration of the region of interest SV.

The processing unit 130 may be configured to form vector information based on the Doppler mode ultrasound data provided from the ultrasound data acquiring unit 120, at step S1506 in FIG. 15. That is, the processing unit 130 may form the vector information corresponding to motion (i.e., velocity and direction) of the target object based on the Doppler mode ultrasound data.

Generally, when the transmission direction of the ultrasound signals is equal to the reception direction of the ultrasound echo signals and a Doppler angle is θ, the following relationship may be established:

$$X cos θ = \frac{{C}_{0} {f}_{d}}{2 {f}_{0}} \qquad ( 1 )$$

In equation 1, X represents a reflector velocity (i.e., velocity of target object), C₀ represents a sound speed in the living body, f_{d} represents a Doppler shift frequency, and f₀ represents an ultrasound frequency.

The Doppler shift frequency f_{d} may be calculated by the difference between a frequency of the ultrasound signals (i.e., transmission beam) and a frequency of the ultrasound echo signals (i.e., reception beam). Also, the velocity component Xcosθ projected to the transmission direction may be calculated by the equation 1.

When the transmission direction of the ultrasound signals (i.e., transmission beam) is different to the reception direction of the ultrasound echo signals (i.e., reception beam), the following relationship may be established:

$$X cos {θ}_{T} + X cos {θ}_{R} = \frac{{C}_{0} {f}_{d}}{{f}_{0}} \qquad ( 2 )$$

In equation 2, θ_{T} represents an angle between the ultrasound signals (i.e., transmission beam) and the blood flow, and θ_{R} represents an angle between the ultrasound echo signals (i.e., reception beam) and the blood flow.

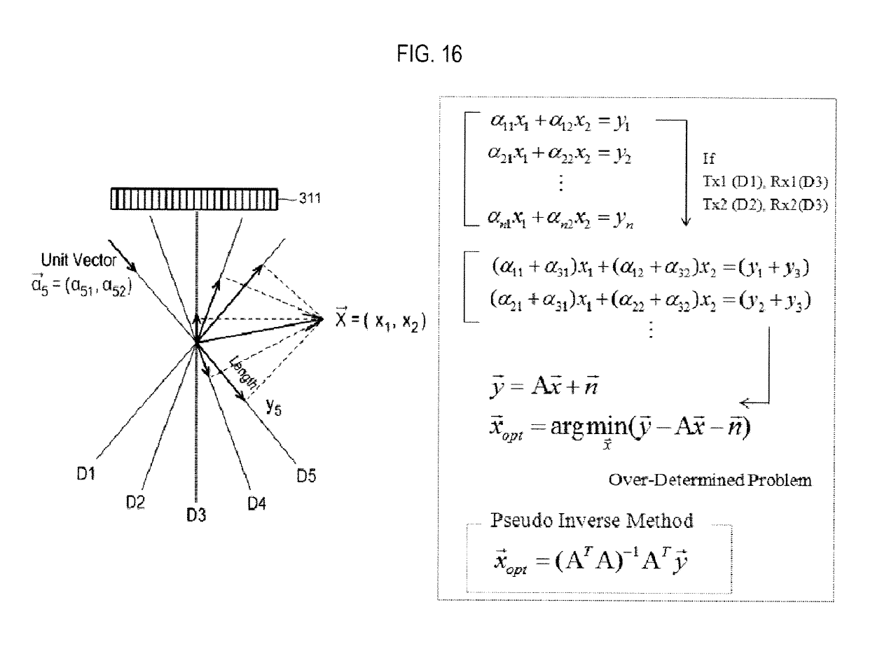

FIG. 16 is a schematic diagram showing an example of the transmission directions, the reception directions, the vector information and an over-determined problem. Referring to FIG. 16, when the ultrasound signals (i.e., transmission beam) are transmitted in a first direction D1 and the ultrasound echo signals (i.e., reception beam) are received in the first direction D1, the following relationship may be established:

\vec{α}\vec{X}=α₁₁x₁+α₁₂x₂=y₁=X cos θ (3)

In equation 3, \vec{α}₁=(α₁₁, α₁₂) represents a unit vector of the first direction D1, \vec{X}=(x₁, x₂) represents variables, and y₁ is calculated by equation 1.

When the ultrasound signals (i.e., transmission beam) are transmitted in a second direction D2 and the ultrasound echo signals (i.e., reception beam) are received in a third direction D3, the following relationship may be established:

(α₂₁+α₃₁)x₁+(α₂₂+α₃₂)x₂=(y₂+y₃)=X cos θ₂+X cos θ₃ (4)

Equations 3 and 4 assume a two-dimensional environment. However, equations 3 and 4 may be expanded to a three-dimensional environment. That is, when expanding equations 3 and 4 to the three-dimensional environment, the following relationship may be established:

α₁₁x₁+α₁₂x₂+α₁₃x₃=y (5)

In the case of the two-dimensional environment (i.e., two-dimensional vector), at least two equations are required to calculate the variables x₁ and x₂. For example, when the ultrasound signals (i.e., transmission beam) are transmitted in the third direction D3 and the ultrasound echo signals (i.e., reception beam) are received in the second direction D2 and a fourth direction D4 as shown in FIG. 16, the following equations may be established:

(α₃₁+α₂₁)x₁+(α₃₂+α₂₂)x₂=(y₃+y₂)

(α₃₁+α₄₁)x₁+(α₃₂+α₄₂)x₂=(y₃+y₄) (6)

The vector \vec{X}=(x₁, x₂) may be calculated by the equations of equation 6.

When the reception beam-forming is performed in at least two angles (i.e., at least two reception directions), at least two equations may be obtained and represented as the over-determined problem, as shown in FIG. 16. The over-determined problem is well known in the art. Thus, it has not been described in detail so as not to unnecessarily obscure the present disclosure. The over-determined problem may be solved by a pseudo inverse method, a weighted least square method and the like based on noise characteristics added to the Doppler shift frequency. That is, M×N equations may be obtained by M transmission directions and the reception beam-forming of N reception directions at every transmission.

The processing unit 130 may be configured to form the Doppler information corresponding to the motion of the target object, at step S1508 in FIG. 15. In one embodiment, the Doppler information may include at least one of a Doppler spectrum image and a Doppler sound corresponding to the motion (i.e., velocity and direction) of the target object.

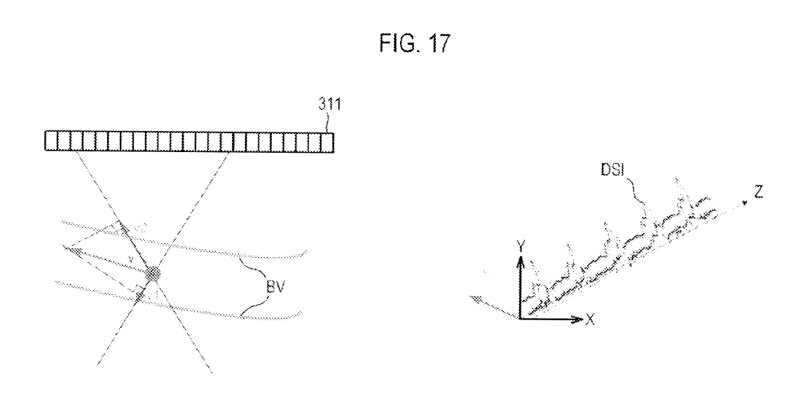

As one example, the processing unit 130 may be configured to form the Doppler information including a Doppler spectrum image corresponding to the motion (i.e., velocity and direction) of the target object based on the vector information. More particularly, the processing unit 130 may form Doppler spectrum corresponding to the velocity of the target object based on the vector information. The processing unit 130 may further set the direction of the target object as a direction of the Doppler spectrum based on the vector information to form the Doppler spectrum image DSI corresponding to the motion (i.e., velocity and direction) of the target object, as shown in FIG. 17. That is, the processing unit 130 may form the Doppler information including the Doppler spectrum image DSI corresponding to the motion (i.e., velocity and direction) of the target object. In FIG. 17, the X-Y axis represents the velocity and the direction of the target object, and the Z axis represents time.

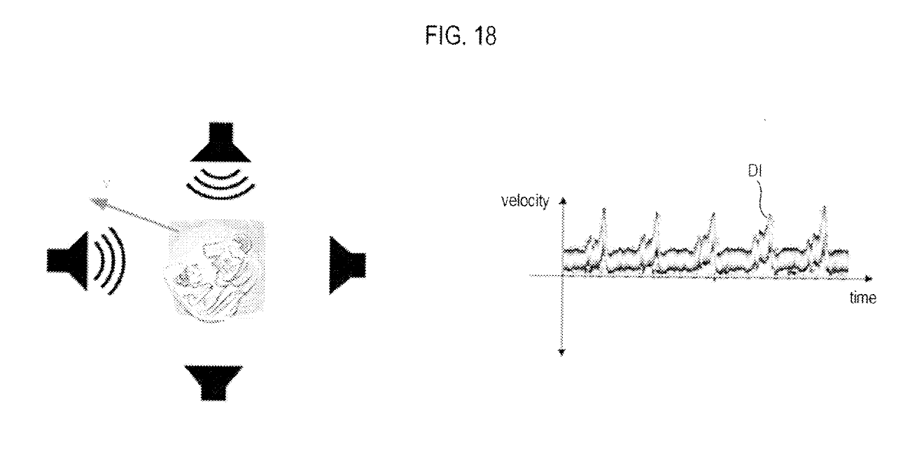

As another example, the processing unit 130 may be configured to form the Doppler information including a Doppler spectrum image corresponding to the velocity of the target object and Doppler sound corresponding to the motion of the target object. More particularly, the processing unit 130 may form the Doppler spectrum image DI corresponding to the velocity of the target object based on the vector information, as shown in FIG. 18. The processing unit 130 may further detect a direction corresponding to the vector information from a plurality of directions (e.g., up, down, left and right), as shown in FIG. 18. The processing unit 130 may further form the Doppler sound corresponding to the detected direction based on the vector information.

As yet another example, the processing unit 130 may be configured to form the Doppler information including the Doppler spectrum image and the Doppler sound corresponding to the motion (i.e., velocity and direction) of the target object. More particularly, the processing unit 130 may form Doppler spectrum corresponding to the velocity of the target object based on the vector information. The processing unit 130 may further set the direction of target object as a direction of the Doppler spectrum based on the vector information to form the Doppler spectrum image DSI corresponding to the motion (i.e., velocity and direction) of the target object, as shown in FIG. 17. The processing unit 130 may further detect a direction corresponding to the vector information from a plurality of directions (e.g., up, down, left and right), as shown in FIG. 18. The processing unit 130 may further form the Doppler sound corresponding to the detected direction based on the vector information.

Optionally, the processing unit 130 may be configured to project the Doppler spectrum image corresponding to the motion (i.e., velocity and direction) of the target object to a predetermined plane to thereby form project information.

As one example, the processing unit 130 may project the Doppler spectrum image corresponding to the motion (i.e., velocity and direction) of the target object to a Y-Z plane to thereby form a project image (hereinafter referred to as “project Doppler spectrum image”) as the projection information.

As another example, the processing unit 130 may project the Doppler spectrum image corresponding to the motion (i.e., velocity and direction) of the target object to the Y-Z plane to thereby form the project Doppler spectrum image as the projection information. The processing unit 130 nay further detect velocity information corresponding to the project Doppler spectrum image to form the Doppler sound corresponding to a right-left direction or a up-down direction as the projection information.

Referring back to FIG. 1, the ultrasound system 100 may further include the storage unit 140. The storage unit 140 may store the ultrasound data (i.e., brightness mode ultrasound data and Doppler mode ultrasound data) acquired by the ultrasound data acquiring unit 120. The storage unit 140 may further store the vector information formed by the processing unit 130.

The ultrasound system 100 may further include the output unit 150. The output unit 150 may be configured to output the ultrasound image (i.e., brightness mode image, Doppler spectrum image and project Doppler spectrum image) and the Doppler sound. In one embodiment, the output unit 150 may include the display unit (not shown) and a plurality of speakers (not shown). The display unit may be configured to display the brightness mode image BI formed by the processing unit 130. The display unit may be further configured to display the Doppler spectrum image and the project Doppler spectrum image formed by the processing unit 130. The plurality of speakers may be configured to output the Doppler sound formed by the processing unit 130.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a processing unit configured to form vector information of a target object based on ultrasound data corresponding to the target object, the processing unit being further configured to form Doppler information corresponding to motion of the target object based on the vector information,
wherein the Doppler information includes at least one of a Doppler spectrum image and a Doppler sound.

2. An ultrasound system of claim 1, wherein the processing unit is configured to form the vector information corresponding to a velocity and a direction of the target object in consideration of at least one transmission direction and at least one reception direction corresponding to the at least one transmission direction based on the ultrasound data.

3. The ultrasound system of claim 2, wherein the processing unit is configured to:
form Doppler spectrum corresponding to the velocity of the target object based on the vector information; and
set the direction of the target object as a direction of the Doppler spectrum based on the vector information to form the Doppler spectrum image corresponding to the motion of the target object.

4. The ultrasound system of claim 3, wherein the processing unit is further configured to project the Doppler spectrum image to a predetermined plane to thereby form a project Doppler spectrum image.

5. The ultrasound system of claim 4, wherein the processing unit is further configured to:
detect velocity information corresponding to the project Doppler spectrum image; and
form the Doppler sound based on the detected velocity information.

6. The ultrasound system of claim 2, wherein the processing unit is configured to:
form the Doppler spectrum image corresponding to the velocity of the target object based on the vector information; and
form the Doppler sound corresponding to the velocity and the direction of the target object based on the vector information.

7. The ultrasound system of claim 2, wherein the processing unit is configured to:
form Doppler spectrum corresponding to the velocity of the target object based on the vector information;
set the direction of the target object a direction of the Doppler spectrum based on the vector information to form the Doppler spectrum image; and
form the Doppler sound corresponding to the velocity and the direction of the target object based on the vector information.

8. The ultrasound system of claim 1, further comprising:
an ultrasound data acquiring unit configured to transmit ultrasound signals to a living body including the target object in at least one transmission direction and receive ultrasound echo signals from the living body in ate least one reception direction to acquire the ultrasound data corresponding to the at least one reception direction.

9. The ultrasound system of claim 8, wherein the ultrasound data acquiring unit is configured to:
transmit the ultrasound signals to the living body in a first transmission direction; and
receive the ultrasound echo signals from the living body in a first reception direction and a second reception direction to acquire the ultrasound data corresponding to the respective first and second reception directions.

10. The ultrasound system of claim 8, wherein the ultrasound data acquiring unit is configured to:
transmit the ultrasound signals to the living body in a first transmission direction and a second transmission direction; and
receive the ultrasound echo signals from the living body in a first reception direction to acquire the ultrasound data corresponding to the first reception direction of the respective first and second transmission directions.

11. The ultrasound system of claim 8, wherein the ultrasound data acquiring unit is configured to:
transmit the ultrasound signals to the living body in a first transmission direction and a second transmission direction; and
receive the ultrasound echo signals from the living body in a first reception direction and a second reception direction to acquire the ultrasound data corresponding to the respective first and second reception directions.

12. The ultrasound system of claim 8, wherein the ultrasound data acquiring unit is configured to transmit the ultrasound signals in an interleaved transmission scheme.

13. The ultrasound system of claim 8, wherein the ultrasound signals include plane wave signals or focused signals.

14. A method of providing Doppler information, comprising:
a) forming vector information of a target object based on ultrasound data corresponding to the target object; and
b) forming Doppler information corresponding to motion of the target object based on the vector information,
wherein the Doppler information includes at least one of a Doppler spectrum image and a Doppler sound.

15. The method of claim 14, wherein the step a) comprises:
forming the vector information corresponding to a velocity and a direction of the target object in consideration of at least one transmission direction and at least one reception direction corresponding to the at least one transmission direction based on the ultrasound data.

16. The method of claim 15, wherein the step b) comprises:
forming Doppler spectrum corresponding to the velocity of the target based on the vector information; and
setting the direction of the target object as a direction of the Doppler spectrum based on the vector information to form the Doppler spectrum image corresponding to the motion of the target object.

17. The method of claim 16, wherein the step b) further comprises:
projecting the Doppler spectrum image to a predetermined plane to thereby form a project Doppler spectrum image.

18. The method of claim 17, wherein the step b) further comprises:
detecting velocity information corresponding to the project Doppler spectrum image; and
forming the Doppler sound based on the detected velocity information.

19. The method of claim 15, wherein the step b) comprises:
forming the Doppler spectrum image corresponding to the velocity of the target object based on the vector information; and
forming the Doppler sound corresponding to the velocity and the direction of the target object based on the vector information.

20. The method of claim 15, wherein the step b) comprises:
forming Doppler spectrum corresponding to the velocity of the target object based on the vector information;
setting the direction of the target object a direction of the Doppler spectrum based on the vector information to form the Doppler spectrum image; and
forming the Doppler sound corresponding to the velocity and the direction of the target object based on the vector information.

21. The method of claim 14, further comprising:
transmitting ultrasound signals to a living body including the target object in at least one transmission direction and receiving ultrasound echo signals from the living body in at least one reception direction to acquire the ultrasound data corresponding to the at least one reception direction, prior to performing the step a).

22. The method of claim 21, wherein the step of acquiring the ultrasound data comprises:
transmitting the ultrasound signals to the living body in a first transmission direction; and
receiving the ultrasound echo signals from the living body in a first reception direction and a second reception direction to acquire the ultrasound data corresponding to the respective first and second reception directions.

23. The method of claim 21, wherein the step of acquiring the ultrasound data comprises:
transmitting the ultrasound signals to the living body in a first transmission direction and a second transmission direction; and
receiving the ultrasound echo signals from the living body in a first reception direction to acquire the ultrasound data corresponding to the first reception direction of the respective first and second transmission directions.

24. The method of claim 21, wherein the step of acquiring the ultrasound data comprises:
transmitting the ultrasound signals to the living body in a first transmission direction and a second transmission direction; and
receiving the ultrasound echo signals from the living body in a first reception direction and a second reception direction to acquire the ultrasound data corresponding to the respective first and second reception directions.

25. The method of claim 21, wherein the ultrasound signals are transmitted in an interleaved scheme.

26. The method of claim 21, wherein the ultrasound signals include plane wave signals or focused signals.
